(19) United States
(12) Patent Application Publication
(10) Pub. No.: US 2003/0072790 A1
(43) Pub. Date: Apr. 17, 2003

(54) Biodegradable porous devices for tissue engineering

(75) Inventors: Chin-Chin Tsai, Taichung Hsien (TW); Hsi-Hsin Shih, Taichung (TW); Huey-Min Lai, Hsinchu (TW)

Correspondence Address: DARBY & DARBY P.C., 805 Third Avenue, New York, NY, 10022, US

(73) Assignee: Industrial Technology Research Institute, Hsinchu (TW)

(21) Appl. No.: 09/982,565

(22) Filed: Oct. 16, 2001

(51) Int. Cl. A61K 9/70; D04H 13/00

(52) U.S. Cl. 424/443; 442/334

(57) ABSTRACT

A biodegradable porous device for tissue engineering is disclosed, which comprises (A) a porous polymeric scaffold comprising a co-continuous phase of a first biodegradable polymer and a second biodegradable polymer which are incompatible with each other, wherein the first biodegradable polymer contains a continuous network of large, interconnected pores, and the second biodegradable polymer contains small, partially interconnected pores; (B) a biodegradable polymer fiber dispersed in, and compatible with the matrix of the first biodegradable polymer; and optionally (C) an active ingredient provided in the polymeric scaffold.

### BACKGROUND OF THE INVENTION

1. Field of the Invention

The present invention relates in general to biodegradable porous devices useful for tissue engineering and regeneration. In particular, this invention relates to a biodegradable porous device comprising a three-dimensional scaffold made up of polymeric components with different degradation rates and pore size distributions.

2. Description of the Related Arts

Synthetic biodegradable polymeric scaffolds have been proposed as a new means of tissue reconstruction and repair. The scaffold serves as both physical support and adhesive substrate for cell growth during in vitro culturing and subsequent in vivo implantation. Scaffolds are utilized to deliver cells to desired sites in the body, to define a potential space for engineered tissue, and to guide the process of tissue development. Cell implantation within scaffolds has been explored for the regeneration of skin, nerve, liver, pancreas, cartilage and bone tissue using various biological and synthetic materials.

In an alternative approach, degradable polymeric scaffolds are implanted directly into a patient without prior culturing of cells in vitro. In this case, the initially cell-free scaffolds need to be designed in such a way that cells from the surrounding living tissue can migrate to the scaffold and adhere/infiltrate into it, forming a functional tissue.

A variety of synthetic biodegradable polymers have been utilized to fabricate tissue engineering scaffolds. Poly(glycolic acid) (PGA), poly(lactic acid) (PLA) and their copolymers are the most commonly used synthetic polymers in tissue engineering. However, in principle, any biodegradable polymer that produces non-toxic degradation products can be used. The potential utility of a porous scaffold as a tissue engineering substrate is primarily dependent upon whether it can provide the similar functionality of extracellular matrices (ECM) as in the body. For example, the tissue scaffold must provide a firm substrate to the transplanted cells and often must be configured into shapes similar to those of the tissue to be repaired.

FIG. 1 is a scanning electron micrograph (SEM) of a prior art tissue scaffold (Gao et al., J. Biomed. Mater. Res., 42, pp. 417-424, 1998), which utilizes non-woven PGA meshes to provide scaffolding for cells to grow on. The non-woven fiber network provides a large open space that is ideal for cell seeding, cell growth, and the production of extra-cellular matrices (ECM). However, the structural rigidity and stability of such scaffolds are limited. Furthermore, even though growth factors can be loaded in the scaffold by attachment to fibers, they cannot be released in a controlled fashion.

FIG. 2 is a SEM photograph of another prior art tissue scaffold comprising a highly porous, open-pore matrix with uniform pore size (Dagalakis et al., J. Biomed. Mater. Res., 14, pp. 511-528, 1980). The high interconnectivity of the pores allows for efficient transport of nutrient and waste product. One problem with this scaffold structure is that the biodegradation kinetics of the scaffold are fixed, and cannot be regulated to accommodate the rapidity of the cell growth. Another problem is that the interconnecting structures can be destroyed due to insufficient rigidity, thus limiting the transport of nutrients and product wastes.

FIG. 3 is a SEM photograph of a further prior art tissue scaffold with a bimodal pore distribution (Levene et al., U.S. Pat. No. 6,103,255). The large pores provide sufficient open space for cell growth while the small pores forming channels between the large pores facilitate the transportation of nutrient and waste product. However, because the scaffold is made of a single material, the bioavailability is somehow limited by its single degradation pattern.

There remains a need in the art for a better architecture for tissue scaffolds.

### SUMMARY OF THE INVENTION

An object of the invention is to provide biodegradable porous devices which can better mimic the extracellular matrices of the body by providing scaffolds with controllable biodegradation kinetics.

Another object of the invention is to provide biodegradable porous devices which allow retention of interconnected pore networks as the cell populations grow.

A further object of the invention is to provide biodegradable porous devices which can be loaded with active substances for subsequent release in a controlled fashion.

These objects are accomplished by providing a biodegradable porous device comprising (A) a polymeric porous scaffold comprising a co-continuous phase of a first biodegradable polymer and a second biodegradable polymer which are incompatible with each other, wherein the first biodegradable polymer contains a continuous network of large, interconnected pores, and the second biodegradable polymer contains small, partially interconnected pores; (B) a biodegradable polymer fiber dispersed in, and compatible with the matrix of the first biodegradable polymer; and optionally (C) an active substance provided in the polymeric scaffold.

According to a first feature of the invention, the porous scaffold is composed of two polymer matrices having different degradation rates and pore morphologies so as to accommodate the biodegradation kinetics of the scaffold to the rapidity of the cell growth.

According to a second feature of the invention, a biodegradable polymer fiber is dispersed in the polymer matrices of the scaffold to increase the mechanical rigidity and to facilitate the cell attachment.

According to a third feature of the invention, an active ingredient such as a growth factor is loaded into pores of the scaffold so that it can be released in a controlled fashion by adjusting the degradation rate of the scaffold.

### BRIEF DESCRIPTION OF THE DRAWINGS

The above and other objects, features, and advantages of the present invention will become apparent from the following detailed description of preferred embodiments of the invention explained with reference to the accompanying drawings, in which:

FIG. 1 is a SEM photograph of a first prior art tissue scaffold;

FIG. 2 is a SEM photograph of a second prior art tissue scaffold;

FIG. 3 is a SEM photograph of a third prior art tissue scaffold;

FIG. 4 is a schematic illustration of the biodegradable porous device of the invention;

FIG. 5 is a diagram of the mass exchange rate as a function of the degradation time, which indicates that the transportation rate of nutrients and waste products increases with degradation time; and

FIG. 6 is a diagram of the structural strengths of the first biodegradable polymer (A), polymer fiber (B), and second biodegradable polymer (C) respectively as a function of the degradation time, which indicates that the first biodegradable polymer (A) has a highest degradation rate, and that the second biodegradable polymer (C) is the main support for the scaffold when the other two gradually dissolve as the cell grows.

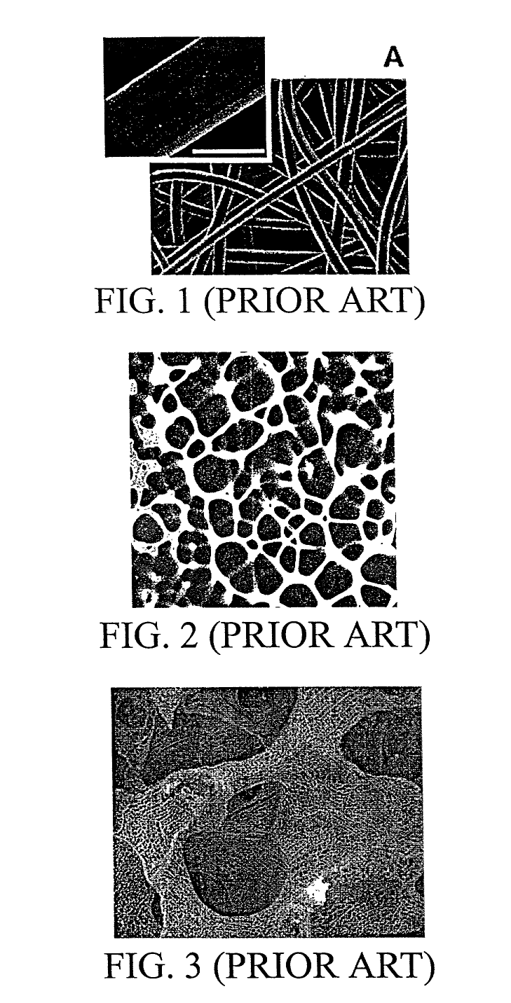

### REFERENCE NUMERALS IN THE DRAWINGS

10 first biodegradable polymer

12 large, interconnected pores

14 second biodegradable polymer

16 small, partially interconnected pores

18 biodegradable polymer fiber

20 active ingredient

### DESCRIPTION OF THE PREFERRED EMBODIMENTS

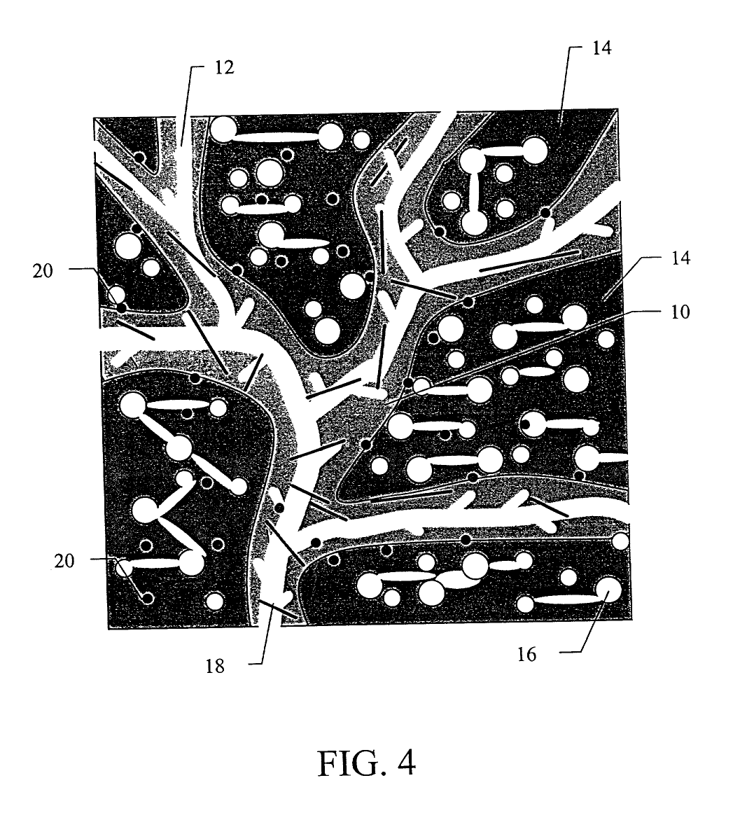

The biodegradable porous device of the invention is described in more detail by referring to the schematic illustration of FIG. 4. A porous polymer scaffold is illustrated having a co-continuous phase of a first biodegradable porous polymer 10 and a second biodegradable porous polymer 14 which are incompatible with each other. A biodegrade polymer fiber 18 compatible with the first biodegradable polymer 10 is uniformly dispersed in its matrix. The biodegradation rate of the first polymer 10 must be higher than that of the second polymer 14 and the polymer fiber 18. Optionally, an active ingredient 20 may be provided predominately in the matrix of the second biodegradable polymer 14 for subsequent release in a controlled fashion as the biodegradable polymer dissolves due to contact with bodily tissue and fluids.

The porous polymer scaffold provides a bimodal pore distribution of large and small pore size. The large pores 12 which have an average pore diameter between about 30 and 250 μm are evenly distributed in the matrix of the first biodegradable polymer 10. The large pores 12 are of sufficient size to form a highly interconnected network. The small pores 16 which have an average pore diameter between about 1 and 50 μm are partially interconnected, embedded in the matrix of the second biodegradable polymer 14. The large pores 12 provide continuous open channel for diffusion of nutrients and oxygen to the cells, and removal of metabolic waste from the cells. The small pores 16 provide additional space for the formation of functional tissue within in the scaffold. In addition, the small pores 16 can be loaded with active substances 20 such as growth factors to promote cellular tissue ingrowth. Both the first and second polymers contain a high degree of porosity, and particularly, the porosity is higher in the first biodegradable polymer 10 than in the second biodegradable polymer 14. Preferably, the first biodegradable polymer 10 has a porosity greater than about 95%, and the second biodegradable polymer 14 has a porosity of about 85 to 95%.

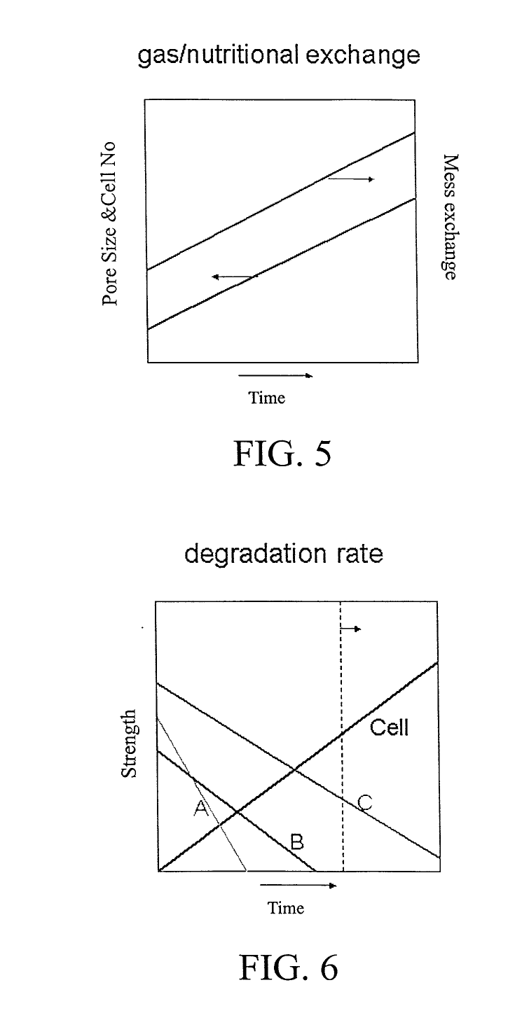

The degradation kinetics of the scaffold can be regulated by varying the types of biodegradable polymers combined, crosslinking density of the polymers, and pore morphologies. In the process of tissue development, the new tissue is first grown on the interconnected pore networks within the first biodegradable polymer 10. The open porosity of the interconnecting structure maximizes diffusion and permits tissue ingrowth into the scaffold. As the first biodegradable polymer 10 gradually disintegrates, the polymer fiber 18 is exposed to facilitate the cell attachment and more space is also released to provide more efficient transport of nutrient and waste product for the increased cell mass (FIG. 5). Meanwhile, the structural rigidity of the scaffold is sustained by the second biodegradable polymer 14, which has a lower degradation rate than the first biodegradable polymer 10 (FIG. 6).

When the first polymer 10 completely disintegrates and its original space is replaced by the newly-grown tissue, the polymer fiber 18 acts as reinforced fiber to provide extra mechanical strength to the scaffold, preventing it from collapse. As a consequence, most of the interconnected pore networks can be preserved for continued transport of nutrient and waste product.

At the final stage of the tissue development, the second biodegradable polymer 18 begins to dissolve for the accommodation of a large number of cells and to ensure sufficient transport of nutrients and waste products. Meanwhile, the active substances loaded in the small pores 16 is released to facilitate the growth and maintenance of the tissues. As the transplanted cell populations grow and the cells function normally, they begin to secrete their own ECM (extracellular matrices) support. Ideally, the polymer is completely resorbed over time, leaving only the newly-formed tissue.

Polymers that are suitable for use in the invention are substantially biodegradable, non-toxic and physiologically compatible. Suitable biodegradable polymers include proteins such as collagen, gelatin, or any other animal or plant proteins; polysaccharides such as hyaluronic acid, chitin, chitosan, and the like; synthetic polymers such as polyvinyl alcohol (PVA), polyglycolic acid (PGA), polylactic acid (PLA), poly(glycolic-co-lactic acid) (PLGA), or polycaprolactone (PCL). A mixture or copolymer of the above is also suitable for use. The most preferred pair of the first and second biodegradable polymer 10, 14 consist of gelatin and collagen, while the polymer fiber 18 is most preferably a synthetic fiber such as a PGA fiber.

Active ingredients suitable for use with the present invention include biologically or pharmaceutically active compounds. Examples of biologically active compounds include cell attachment mediators, such as the peptide containing variations of the “RGD” integrin binding sequence known to affect cellular attachment, biologically active ligands, and substances that enhance or exclude particular varieties of cellular or tissue ingrowth. Such substances include, for example, osteoinductive substances, such as bone morphogenic proteins (BMP), epidermal growth factor (EGF), fibroblast growth factor (FGF), platelet-derived growth factor (PDGF), insulin-like growth factor (IGF-I and II), TGF-β and the like.

Examples of pharmaceutically active compounds include, for example, acyclovir, cephradine, malfalen, procaine, ephedrine, adriomycin, daunomycin, plumbagin, atropine, quanine, digoxin, quinidine, biologically active peptides, chlorin e₆, cephalothin, proline and proline analogues such as cis-hydroxy-L-proline, penicillin V, aspirin, ibuprofen, steroids, nicotinic acid, chemodeoxycholic acid, chlorambucil, and the like. Therapeutically effective dosages may be determined by either in vitro or in vivo methods. For each particular active substance, individual determinations may be made to determine the optimal dosage required. The determination of effective dosage levels, that is, the dosage levels necessary to achieve the desired result, will be within the ambit of one skilled in the art. The release rate of the active substance may also be varied within the routine skill in the art to determine an advantageous profile, depending on the therapeutic conditions to be treated.

The porous polymer scaffolds are shaped into articles for tissue engineering and regeneration applications, including reconstructive surgery. The porous polymer scaffolds may also be molded to form external scaffolding for the support of in vitro culturing of cells for the creation of external support organs. The scaffolds may also be used in transplantation as a matrix for dissociated cells.

While the invention has been particularly shown and described with reference to the preferred embodiment thereof, it will be understood by those skilled in the art that various changes in form and details may be made without departing from the spirit and scope of the invention.

What is claimed is:

1. A biodegradable porous device, comprising:
a porous polymeric scaffold comprising a co-continuous phase of a first biodegradable polymer and a second biodegradable polymer which are incompatible with each other, wherein the first biodegradable polymer contains a continuous network of large, interconnected pores, and the second biodegradable polymer contains small, partially interconnected pores;
a biodegradable polymer fiber dispersed in, and compatible with the matrix of the first biodegradable polymer; and optionally
an active ingredient provided in the polymeric scaffold.

2. The biodegradable porous device as claimed in claim 1, wherein the first biodegradable polymer has a higher biodegradation rate than the second biodegradable polymer and the biodegradable polymer fiber.

3. The biodegradable porous device as claimed in claim 1, wherein the first biodegradable polymer has a higher porosity than the second biodegradable polymer.

4. The biodegradable porous device as claimed in claim 3, wherein the first biodegradable polymer has a porosity greater than about 95%, and the second biodegradable polymer has a porosity of about 85 to 95%.

5. The biodegradable porous device as claimed in claim 1, wherein the large pores have an average pore diameter between about 30 and 250 μm, and the small pores have an average pore diameter between about 1 and 50 μm.

6. The biodegradable porous device as claimed in claim 1, wherein the first biodegradable polymer is selected from the group consisting of proteins, polysaccharides, synthetic materials, and mixtures or copolymers thereof.

7. The biodegradable porous device as claimed in claim 1, wherein the second biodegradable polymer is selected from the group consisting of proteins, polysaccharides, synthetic materials, and mixtures or copolymers thereof.

8. The biodegradable porous device as claimed in claim 1, wherein the biodegradable polymer fiber is selected from the group consisting of proteins, polysaccharides, synthetic materials, and mixtures or copolymers thereof.

9. The biodegradable porous device as claimed in claim 1, wherein the active ingredient is provided predominately in the matrix of the second biodegradable polymer.

10. The biodegradable porous device as claimed in claim 1, wherein the polymeric scaffold comprises an effective amount of a biologically active substance that either promotes or prevents a particular variety of cellular tissue ingrowth.

11. The biodegradable porous device as claimed in claim 1, wherein the polymeric scaffold comprises an effective amount of a pharmaceutically active compound.

12. A biodegradable porous device, comprising:
a porous polymeric scaffold comprising a co-continuous phase of a first biodegradable polymer and a second biodegradable polymer which are incompatible with each other, wherein the first biodegradable polymer contains a continuous network of large, interconnected pores with an average pore diameter between about 30 and 250 μm, the second biodegradable polymer contains small, partially interconnected pores with an average pore diameter between about 1 and 50 μm, and the first biodegradable polymer has a higher biodegradation rate than the second biodegradable polymer;
a biodegradable polymer fiber dispersed in, and compatible with the matrix of the first biodegradable polymer; and
an active ingredient provided predominately in the matrix of the second biodegradable polymer.

13. The biodegradable porous device as claimed in claim 12, wherein the first biodegradable polymer has a higher porosity than the second biodegradable polymer.

14. The biodegradable porous device as claimed in claim 13, wherein the first biodegradable polymer has a porosity greater than about 95%, and the second biodegradable polymer has a porosity of about 85 to 95%.

15. The biodegradable porous device as claimed in claim 12, wherein the first biodegradable polymer is gelatin and the second biodegradable polymer is collagen.

16. The biodegradable porous device as claimed in claim 15, wherein the biodegradable polymer fiber is made of a synthetic polymer selected from the group consisting of polyvinyl alcohol (PVA), polyglycolic acid (PGA), polylactic acid (PLA), poly(glycolic-co-lactic acid) (PLGA), and polycaprolactone (PCL).

17. The biodegradable porous device as claimed in claim 12, wherein the polymeric scaffold comprises an effective amount of a biologically active substance that either promotes or prevents a particular variety of cellular tissue ingrowth.

18. The biodegradable porous device as claimed in claim 12, wherein the polymeric scaffold comprises an effective amount of a pharmaceutically active compound.
